SEQUENCE LISTING (12 sequences)

SEQ ID NO: 1 - DNA, 15 bp, Artificial Sequence
  HPV 16 primer
agggccaactaaatg

SEQ ID NO: 2 - DNA, 19 bp, Artificial Sequence
  HPV 16 primer
ctgcttttatactaaccgg

SEQ ID NO: 3 - DNA, 24 bp, Artificial Sequence
  BRCA1 primer
gaagttgtcattttataaaccttt

SEQ ID NO: 4 - DNA, 22 bp, Artificial Sequence
  BRCA1 primer
tgtcttttcttccctagtatgt

SEQ ID NO: 5 - DNA, 20 bp, Artificial Sequence
  p53 primer
tactcccctgccctcaacaa

SEQ ID NO: 6 - DNA, 20 bp, Artificial Sequence
  p53 primer
catcgctatctgagcagcgc

SEQ ID NO: 7 - DNA, 20 bp, Artificial Sequence
  Chlamydia Trachomatis primer
tagtaactgccacttcatca

SEQ ID NO: 8 - DNA, 23 bp, Artificial Sequence
  Chlamydia Trachomatis primer
ttccccttgtaattcgttgttgc

SEQ ID NO: 9 - DNA, 20 bp, Artificial Sequence
  Mycobacterium tuberculosis primer
tccgctgccagtcgtcttcc

SEQ ID NO: 10 - DNA, 20 bp, Artificial Sequence
  Mycobacterium tuberculosis primer
gtcctcgcgagtctaggcca

SEQ ID NO: 11 - DNA, 23 bp, Artificial Sequence
  Mycoplasma pneumoniae primer
caagccaaacacgagctccggcc

SEQ ID NO: 12 - DNA, 26 bp, Artificial Sequence
  Mycoplasma pneumoniae primer
ccagtgtcagctgtttgtccttcccc

(19) United States
(12) Patent Application Publication
(10) Pub. No.: US 2004/0241654 A1
(43) Pub. Date: Dec. 2, 2004

(54) Cytological specimen loaded filter paper and an efficient method of using said paper for dry collection, transportation, and storage to screen for infection using PCR

(75) Inventors: Bhudev C. Das, New Delhi (IN); Suresh Hedau, New Delhi (IN); V. Gopalkrishna, New Delhi (IN); Sanjay Katiyar, New Delhi (IN); U. Kailash, New Delhi (IN)

Correspondence Address: OLIFF & BERRIDGE, PLC, P.O. BOX 19928, ALEXANDRIA, VA, 22320, US

(73) Assignee: Indian Council of Medical Research, New Delhi (IN)

(21) Appl. No.: 10/444,988

(22) Filed: May 27, 2003

(51) Int. Cl. C12Q 1/68

(52) U.S. Cl. 435/6

(57) ABSTRACT

The present invention relates to a cytological specimen loaded filter paper useful for dry collection, transportation, and storage of cytological specimens at temperature ranging between 4° C. to 50° C. to screen for identification of gene sequence of pathogens responsible for infection using PCR, wherein the said loaded-paper is workable for about fifteen years from the time of loading for large scale screening especially for population from distant places, and also, a simple, rapid, safe, and cost-effective filter-paper method of dry collection, transportation, and storage of cytological specimens at temperature ranging between 4° C. to 50° C. to screen for pathogenic genomes and cellular genes using PCR.

### FIELD OF THE PRESENT INVENTION

The present invention relates to a cytological specimen loaded filter paper useful for dry collection, transportation, and storage of cytological specimens at temperature ranging between 4° C. to 50° C. to screen for identification of gene sequences of pathogens responsible for infection using PCR.

### BACKGROUND AND PRIOR ART REFERENCES

Cancer of the uterine cervix is the most common malignant tumour ii women world-wide and represents a major public health problem in India and south-east Asia. In India, c. 100 000 women develop this cancer every year [1,2], contributing c. 16% of the global annual incidence [3]. Human papillomaviruses (HPVs) have emerged as major pathogens associated with this disease [4-6]. Around 20 of the 100 or more different HPV types thus far identified are associated with cancers of the lower genital tract, whereas other types cause warts and other diseases in different epithelial organ sites [7, 8]. Up to 98% of cervical cancers may be positive for HPV [9]. HPV infections in the general population are highly prevalent as clinically latent infections. Individuals carrying ‘high-risk’ HPVs (e.g., types 16 and 18) show a high rate of progression of dysplasia to invasive cancer of the cervix [5,10-12]. Thus, reliable diagnosis of HPVs may facilitate early identification of women at increased risk of developing cervical cancer.

Furthermore, the Papanicolaou smear test (Pap test), generally employed for cytomorphological diagnosis of early cervical lesions, is not fully reliable. Incorporation of HPV testing along with the Pap test may significantly augment the sensitivity and specificity of primary cervical cancer screening programmes [13, 14]. Diagnosis of HPV relies mainly upon viral genome detection by nucleic acid hybridization or PCR assays of cellular DNA extracted from either cervical scrapes or tumour biopsy specimens. However, these methods suffer from several technical limitations associated with collection, transport and storage of specimens. Scraped cervical cells or tumour biopsies are generally collected in cold PBS solution, transported on ice, and stored at −70° C. or in liquid nitrogen until further processing. The standard procedure of DNA isolation by proteinase K digestion and phenol-chloroform extraction is complex, expensive, hazardous, time-consuming and unsuitable for screening large numbers of specimens at a time.

This paper describes a simple ‘paper smear’ procedure for collecting cervical smear/scrapes or biopsy specimens on to a sterile paper slide made up of Whatman 3MM filter paper which can then be easily air-dried and stored at room temperature. The slides can then be easily transported from the clinic or field to the laboratory. The technique was validated with various other biological specimens. A procedure has been widely used for collecting dry blood spots to screen for various infectious agents [15-19] and metabolic and genetic diseases [20-22]. However, the method of the instant Application is new for collection of scrapped cervical cells and any other cytological or biopsy imprints specimens in dry forms for detection of HPV or host of other pathogens and gene sequences of interest. It is already mentioned in the paper that the specimens can be transported and stored at temperature ranging between 4° C. to 50° C. for several years.

### OBJECTS OF THE PRESENT INVENTION

The main object of the present invention is to develop a cytological specimen loaded filter paper useful for dry collection, transportation, and storage of cytological specimens at temperature ranging between 4° C. to 50° C. to screen for sequence of pathogens using PCR. Another main object of the present invention is to develop a specimen loaded paper loaded with the cytological specimens selected from a group comprising cultured cells, bacterial cells, viral cells, cervical and buccal smears, scrapes, blood, urine, amniotic/ascitic fluid, semen, and bone marrow or needle aspirates, including solid tissue biopsy imprints.

Yet another object of the present invention is to develop a specimen loaded paper workable for about fifteen-twenty years.

Still another object of the present invention is to develop a specimen loaded paper usable for large scale screening especially for population from distant places.

Another main object of the present invention is to develop a simple, rapid, safe, and cost-effective filter-paper method of dry collection, transportation, and storage of cytological specimens at temperature ranging between 4° C. to 50° C. to screen for detection of infection using PCR.

Yet another object of the present invention is to maintain DNA in its native conformation.

### SUMMARY OF THE PRESENT INVENTION

The present invention relates to a cytological specimen loaded filter paper useful for dry collection, transportation, and storage of cytological specimens at temperature ranging between 4° C. to 50° C. to screen for gene sequences of pathogens using PCR, wherein the said loaded-paper is workable for about fifteen years for large scale screening especially for population from distant places, and also, a simple, rapid, safe, and cost-effective filter-paper method of dry collection, transportation, and storage of cytological specimens at temperature ranging between 4° C. to 50° C. to screen for infection using PCR.

### DETAILED DESCRIPTION OF THE PRESENT INVENTION

Accordingly, the present invention relates to a cytological specimen loaded filter paper useful for dry collection, transportation, and storage of cytological specimens at temperature ranging between 4° C. to 50° C. to screen for gene sequence of pathogens using PCR, wherein the said loaded-paper is workable for about fifteen years for large scale screening especially for population from distant places, and also, a simple, rapid, safe, and cost-effective filter-paper method of dry collection, transportation, and storage of cytological specimens at temperature ranging between 4° C. to 50° C. to screen for detection of infection using PCR.

In an embodiment of the present invention, wherein a cytological specimen loaded filter paper useful for dry collection, transportation, and storage of cytological specimens at temperature ranging between 4° C. to 50° C. to screen for gene sequences of pathogens using PCR.

In another embodiment of the present invention, wherein the cytological specimens are selected from a group comprising cultured cells, cervical and buccal smears, scraps, blood, urine, amniotic/ascitic fluid, semen, and bone marrow or needle aspirates, including solid tissue biopsy imprints.

In yet another embodiment of the present invention, wherein the loaded-paper is stored in a sterile airtight bag.

In still another embodiment of the present invention, wherein the loaded-paper is workable for about fifteen years.

In still another embodiment of the present invention, wherein said paper is used for large scale screening especially for population from distant places.

In another main embodiment of the present invention, A simple, rapid, safe, and cost-effective filter-paper method of dry collection, transportation, and storage of cytological specimens at temperature ranging between 4° C. to 50° C. to screen for detection of infection using PCR, said method comprising the steps of:

1. applying the cytological specimen on to an optionally pre-treated filter paper,

2. drying the paper comprising the specimen,

3. storing the paper of step (b) in a sterile airtight bag,

4. adding small piece of the dried paper of step(c) into distilled water to obtain a solution,

5. boiling the solution for a time duration ranging between 4-8 minutes,

6. concentrating the solution to a range of one-fourth to one-fifth of the original volume to obtain a concentrate comprising DNA,

7. adding PCR-mix to the concentrate,

8. conducting direct-PCR on the concentrate, and

9. screening for the infection using PCR product on electrophoresis.

In still another embodiment of the present invention, wherein the method helps early identification of a disease condition.

In still another embodiment of the present invention, wherein the filter paper is 3MM filter paper.

In still another embodiment of the present invention, wherein boiling the solution for about 5 minutes for elusion of DNA.

In still another embodiment of the present invention, wherein boiling is in microwave at about 360 W.

In still another embodiment of the present invention, wherein DNA is stable in the paper for about fifteen years.

In still another embodiment of the present invention, wherein said method can be used for large scale screening especially for population from distant places.

In still another embodiment of the present invention, wherein about 90% of the DNA content is retrieved from the paper smears.

In still another embodiment of the present invention, wherein DNA maintains its native form.

In still another embodiment of the present invention, wherein the paper is pretreated with antibiotics, fungicides, and denaturants.

In still another embodiment of the present invention, wherein said method can be employed for all types of biological specimens comprising cultured cells, cervical and buccal smears, scrapes, blood, urine, amniotic/ascitic fluid, semen, and bone marrow or needle aspirates, including solid tissue biopsy imprints.

In still another embodiment of the present invention, wherein the bag is preferably sealed polythene bag.

Human papillomaviruses (HPVs) are major pathogens associated with the development of cancer of the uterine cervix, the most common malignant tumour of women world wide. Reliable diagnosis of HPV infection, particularly the ‘high-risk’ types (16/18), may facilitate early identification of ‘high-risk’ populations for developing cervical cancer and may augment the sensitivity and specificity of primary cervical cancer screening programmes by complementing the conventional Pap test. A simple paper smear method has been developed for dry collection, transport and storage of cervical smears/scrapes at room temperature for subsequent detection of HPV DNA by PCR assay. Imprint biopsies, blood and fine-needle aspirates were also collected by this method. The cervical scrapes or other body fluids were smeared (within 0.5-1 cm diameter) and dried on to sterile small slides made of Whatman 3MM filter paper, and stored individually at room temperature or at 4° C. A small piece (2-3 mm) of the paper smear was punched or cut out with a sterile surgical blade, boiled in an eppendorf tube containing 50 μl of distilled water for about 5 min and used directly for PCR amplification. The quality and quantity of DNA derived from paper smears and the results of PCR amplifications for HPV type 16, BRCAI and p53 genes were identical to those obtained from the same samples following standard collection in PBS, storage (−70° C.) and phenol-chloroform-based DNA extraction. DNA was stable in the paper smears for time duration of about fifteen years, whether stored at room temperature or at 4° C. This method is simple, rapid and cost-effective, and can be effectively employed for large-scale population screening, especially for regions where the specimens are to be transported from distant places to the laboratory.

Dry ‘paper Smear’ for Rapid HPV Testing

Infection of specific ‘high risk’ types of Human Papillomavirus (HPV), a small DNA virus is considered to be an etiologic agent that causes cancer of the uterine cervix in women. Cervical cancer is curable if detected early. Therefore, reliable early detection of these cancer-causing high risk HPV types would facilitate early identification of high risk populations for developing cervical cancer. Conventional cytomorphological diagnosis by Pap test commonly employed for the purpose is not fully reliable and is not universally available at least in developing countries including India. In absence of reliable type-specific serological tests, diagnosis of HPV relies mainly upon detection of viral genomes by molecular methods.

The most commonly used highly sensitive method world over is nucleic acid amplification method or polymerase chain reaction (PCR). This or any other molecular methods require collection, transport of cervical scrapes or tumor biopsy specimens on ice and their storage at −70° C. deep freezer or liquid nitrogen until further processing. The standard DNA extraction by proteinase K digestion and phenol-chloroform extraction is complex, expensive, time-consuming and hazardous. This is certainly not suitable for screening large number of specimens at a time.

A simple and inexpensive dry collection method has been developed for collection of scraped cervical cells/smear or biopsy imprints on to a small sterile paper slide made up of 3MM Whatman filter paper. Smearing of cervical scrape is done 0.5 to 1.0 cm diameter in the middle of the filter paper slide (see flow chart). Tumour biopsies are either imprinted or tumour cell suspension is blotted on to the paper slide. Several such clinical specimens such as buccal smear, blood, fine needle aspirates, urine, semen, bone marrow or any type of body fluid/cell/bacterial cultures etc. can be collected for screening pathogens and gene sequences including for forensic purposes. The air-dried paper smears are then put individually in a ziplock polythene bag and can be stored either at room temperature or at 4° C. in a fridge. A small 2-3 mm piece of the paper smear is cut out by a strile surgical blade and boiled in an eppendorf tube (0.5 ml) containing 50 μl of distilled water for 5 minutes.

Boiling is essential in a microwave oven to destroy PCR inhibitory factors. DNA thus eluted is directly used for PCR amplification in the same tube after addition of PCR amplification mix. The paper smear can be stored at room temperature for more than about 15-20 years without any effect on the quality of DNA or PCR amplification. Since this dry paper smear does not require refrigeration for storage, it is easier to transport from distant places/fields/clinics to the laboratory. These can easily be shipped/sent through ordinary post. So far more than about 1000 paper smears have been tested and the results show 100% correlation with the conventional/standard procedures of sample collection, DNA extraction and PCR amplification.

This method is also suitable for extraction of larger amount of DNA required for additional molecular genetics studies such as RFLP, (restriction fragment length polymorphism), cloning, sequencing etc. The method has been tested for detection of p53, BRCAI and BRCA2 genes besides HPV. Fine needle aspirates and other body fluids have been used for detection of Mycobacterium tuberculosis, Clamydia trachomatis and many other reproductive tract infections. This method, in fact, can be employed universally for all kinds of biological specimens for screening genes and pathogens. At present, no such technique is available for screening HPV in cervical smear.

Paper Smear Method for Rapid HPV Testing

Applicants have developed a novel paper smear method for dry collection, transportation, and storage at room temperature of cervical smears, coupled with a single step DNA extraction procedure, which they say will provide a cost-effective and reliable alternative to standard tests.

Cervical cells are collected on small sterile slides made of filter paper. A 2-3 mm piece of the paper smear is punched or cut out with a sterile surgical blade and sent for testing. The smears are then boiled in distilled water in a microwave oven and used directly for PCR amplification to defect HPV.

High-risk HPV, are associated with cancers of the lower genital tract. The method can be used for all types of biological specimens such as cultured cells, baccal smears, blood and needle aspirates including solid tissue biopsv imprints.

The study, included an analysis of more than 500 paper smears (including biopsy and fine needle aspiration cytology samples from women with breast carcinoma) from various hospitals—many of them were packed in auto-seal polythene bags and sent by post. The quality and quantity of DNA derived from paper smears were just as good as that obtained by standard collection and phenol-chloroform-based DNA extraction. The results of PCR amplification for HPV type 16, BRAC1 and p53 genes were also equivalent.

The most difficult aspect of any DNA study is collecting biological specimens on ice and storing them at an ultra low temperature (−70° C. or liquid nitrogen). This technique does away with all this. The samples do not get spoiled even when stored at high temperatures for over 15 years.

Using both the Pap smear and the HPV paper smear to screen women as early as possible would be of immense value in improving the management of cervical cancer. In India, about 100 000 women develop cancer of the uterine cervix every year and about 98% of these cancers are HPV positive.

Materials and Methods

Study Subjects and Specimens

Four different types of biological specimens were collected: (i) 50 cervical scrapes and (ii) 50 tumour biopsy specimens, for detection of HPV 16 DNA sequences, (iii) fine-needle aspirate cells (FNACs) from 30 women with breast carcinomas for detection of the breast cancer susceptibility gene BRCAI (exon 2) and the p53 gene (exon 5), and (iv) peripheral venous blood from 20 normal healthy individuals for detection of the same p53 gene exon.

Specimen Collection, Transport and Storage

Cervical scrapes and tumour biopsies were obtained from women with cervical dysplasias and carcinomas, respectively, from the Gynecology Outpatient Department and Cancer Clinic of Lok Nayak Hospital, New Delhi. Scraped cervical cells obtained by Ayer's spatula were first smeared on to a 3MM Whatman paper cut to the size of a small glass slide (5 cm×2 cm). Smearing was done within a 0.5-1.0 cm diameter in the middle of the filter-paper slide. The spatula and the rest of the scraped cell materials were transferred to a 15-ml collection vial containing 5 ml of PBS on ice and then stored at −70° C. Tumour biopsies were either imprinted or the tumour cell suspension was blotted on to the paper slide.

The remaining biopsy was collected in PBS and stored at −70° C. for comparison. FNACs (5-10 μl from breast cancer patients and heparinised blood (5-10 μl) from healthy individuals were spotted as paper smears and the remaining material was processed by standard procedures. All paper smears performed in duplicate were put into individual auto-seal (ziplock) polythene bags. Half of these bags were stored at room temperature (25-30° C.) and the rest at 4° C. until analysis. Cervical specimens as paper smears were also obtained in envelopes sent by post from distant parts of India. All samples were stored for at least 3-4 weeks before analysis. One set of paper smears was stored for up to 10-15 years at room temperature.

Conventionally, the specimens are collected in cold PBS, stored at −20° C. or −70° C. deep freezer and processed in cold condition (0-4° C.). However, the specimens of the instant Application can be stored at any temperature ranging between 4-50° C.

DNA Extraction and PCR

A small piece (2-3 mm) of the dried paper smear specimens was punched or cut out with a sterile singlecut paper-punching machine or with a new sterile scalpel blade, and transferred to a 0.5-ml microcentifuge tube containing 50 μl of distilled water. This was then boiled for 5 min in a microwave oven (360 W; Bosch). After reduction in volume to 10,ul in a speed-vac concentrator, PCR mix with Taq DNA polymerase (Perkin-Elmer Cetus, Roche, N.J., USA) and primers was added to the tube. Amplification was performed in a 30-,ul reaction mix in a DNA thermal cycler (Perkin-Elmer Cetus) by a protocol described previously [9, 12]. A larger amount of DNA was also extracted from several single punched-out paper smear disks in an eppendorf tube by a non-organic method described previously [23]. DNA extraction from cervical scrapes and tumour biopsies collected in PBS and stored at −70° C. was performed by standard proteinase K digestion and phenol-chloroform methodology [9, 12, 24].

PCR Amplification and Amplicon Detection

Two PCR reactions, one for DNA obtained by standard procedures and another for paper smear-derived DNA, were performed simultaneously for each of the four types of biological specimens collected. Also, PCR was performed separately for the DNA of paper smear samples stored either at room temperature or at 4° C. The primers for HPV 16, BRCAI and p53 (see Table 1 as shown below) were synthesized in an automated DNA Synthesizer (Model 381A; Applied Biosystems, Foster City, Calif., USA) by phosporamidite chemistry and were purified by high-performance liquid chromatography (HPLC). After PCR, 15,ul of the amplified product were run on an ethidium bromide-stained Nusieve agarose 3% gel (FMC Bioproducts, Rockland, USA) and visualised with a UV transilluminator. Every PCR reaction included positive and negative controls. Strict laboratory precautions and control measures [25] were followed to avoid cross-contamination and carry-over in the PCR assay.

Basically, cell concentration in the filter paper is not measured. The routine procedure of smear preparation is done on whole slides but, here it is done on paper slide in a limited space (0.5 to 1 cm diameter) to concentrate cell samples. PCR can amplify as less as one cell if present on the 1-2 mm piece of paper smear which is used for one PCR reaction and one paper smear can be sufficient for 10-12 PCR reactions.

### BRIEF DESCRIPTION OF THE ACCOMPANYING DRAWINGS

FIG. 1 shows flow chart describing steps of DNA extraction PCR amplification using filter paper.

FIG. 2 shows Ethidium bromide-stained agarose gel electrophoresis showing quality and quantity of high mol. wt genomic DNA extracted from cervical scrapes (a, b), fine-needle aspirated breast carcinoma cells (c, d) and blood (e, f). DNA samples shown in upper panels (a, c and e) were extracted by standard phenol-chloroform methods following collection of specimens in cold PBS and storage at −70° C., whereas the DNA samples shown in the lower panels (b, d and f) were from the same samples collected on paper smears and DNA extracted by boiling.

FIG. 3 shows PCR amplification of HPV type 16 in cervical scrapes (a, b) and cervical tumour biopsy specimens (c, d) collected and stored by standard procedures (a, c) and by paper smear methods (b, d). Amplimers of 217 bp are seen in lanes 3, 4 and 7 in both (a) and (b) and lanes 3, 4, 8 and 9 in (c) and (d). Lane 1 in (a) and (b) and lane 2 in (c) and (d) are positive controls. Lane 2 in (a) and (b) and lane 1 in (c) and (d) are negative controls (placental DNA).

FIG. 4 shows PCR amplification of HPV 16 in cervical scrapes collected as paper smears and stored at room temperature for (a) 6 months and (b) I year. Lanes: 1, HaeIII-digested φX174 DNA mol. wt markers; 2, positive control; 3 and 4, HPV-positive samples; 5 and 6, negative samples.

### RESULTS

The quality and quantity of DNA extracted from the dried paper smears by boiling were comparable to those obtained by standard collection and phenol

TABLE 1

Genomes of Pathogenes and Genes Detected by Paper Smear

No. of

| | | | | | |
|---|---|---|---|---|---|
| S. | Genomes/ | Primers | Amplimer | cases | Accession |

| | | | | | | |
|---|---|---|---|---|---|---|
| No | Genes | Location | Primer Sequences | size | studied | number |

| | | | | | | |
|---|---|---|---|---|---|---|
| 1. | HPV 16 | URR | 5′-AGG GCC | 217 bp | 100 | Gene |
| | | | AAC TAA ATG | | | Bank |
| | | | 5′-CTG CTT | | | K02718 |
| | | | TTA TAC TAA | | | |
| | | | CCG G-3′ | | | |
| 2. | BRCA1 | Exon 2 | 5′-GAA GTT GTC | 258 bp | 30 | Gene |
| | | | ATT TTA TAA | | | Bank |
| | | | ACC TTT-3′ | | | L78833 |
| | | | 5′-TGT CTT TTC | | | |
| | | | TTC CCT AGT ATG | | | |
| | | | T-3′ | | | |
| 3. | p53 | Exon 5 | 5′-TAC TCC CCT | 184 bp | 20 | EMBL |
| | | | GCC CTC AAC | | | X54156 |
| | | | AA-3′ | | | |
| | | | 5′-CAT CGC TAT | | | |
| | | | CTG AGC AGC | | | |
| | | | GC-3′ | | | |
| 4. | Chlamydia | Plasmid | 5′-TAG TAA CTG | 201 bp | 50 | Gene |
| | Trachomatis | | CCA CTT CAT | | | Bank |
| | | | CA-3′ | | | Ae001273 |
| | | | 5′-TTC CCC TTG | | | |
| | | | TAA TTC GTT GTT | | | |
| | | | GC-3′ | | | |
| 5. | Mycobacterium | | 5′-TCC GCT GCC | 240 bp | 75 | Gene |
| | tuberculosis | | AGT CGT CTT CC- | | | Bank |
| | | | 3′ | | | AE000516 |
| | | | 5′-GTC CTC GCG | | | |
| | | | AGT CTA GGC CA- | | | |
| | | | 3′ | | | |
| 6. | Mycoplasma | | 5′-CAA GCC | 543 bp | 75 | Gene |
| | pneumoniae | | AAA CAC GAG | | | Bank |
| | | | CTC CGG CC- | | | U00089 |
| | | | 3′ | | | |
| | | | 5′-CCA GTG | | | |
| | | | TCA GCT GTT | | | |
| | | | TGT CCT TCC | | | |
| | | | CC-3′ | | | |

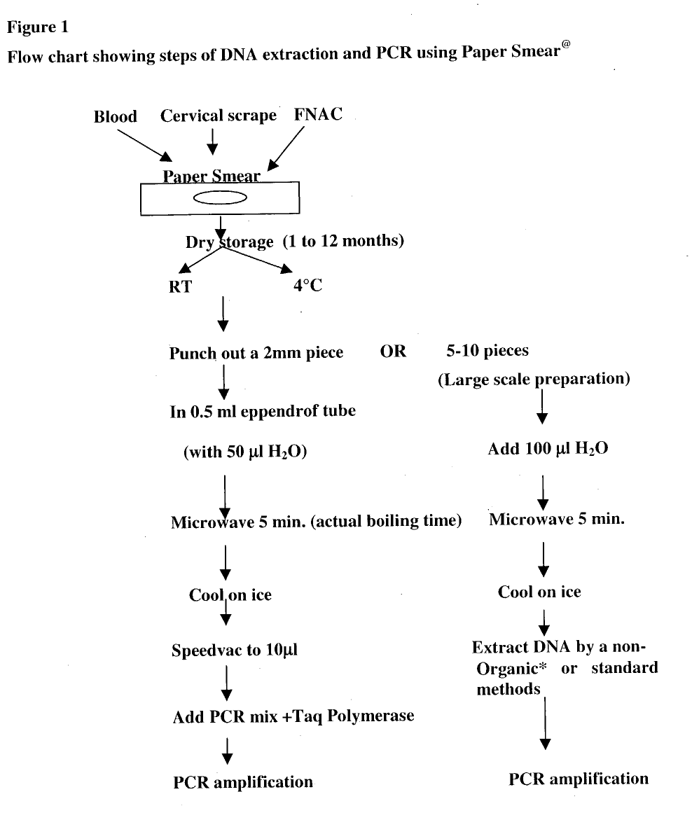

chloroform extractions, as estimated by ethidium bromide-stained agarose gel electrophoresis (FIG. 1). There was also no difference in the quality of DNA isolated from paper smears stored at room temperature or at 4° C. To determine an optimal boiling time for DNA elution from paper smears, single punched-out paper disks were boiled for 2, 5, 7 or 10 min. Microwaving for 5 min (actual boiling time) gave the best results in terms of PCR signal generated.

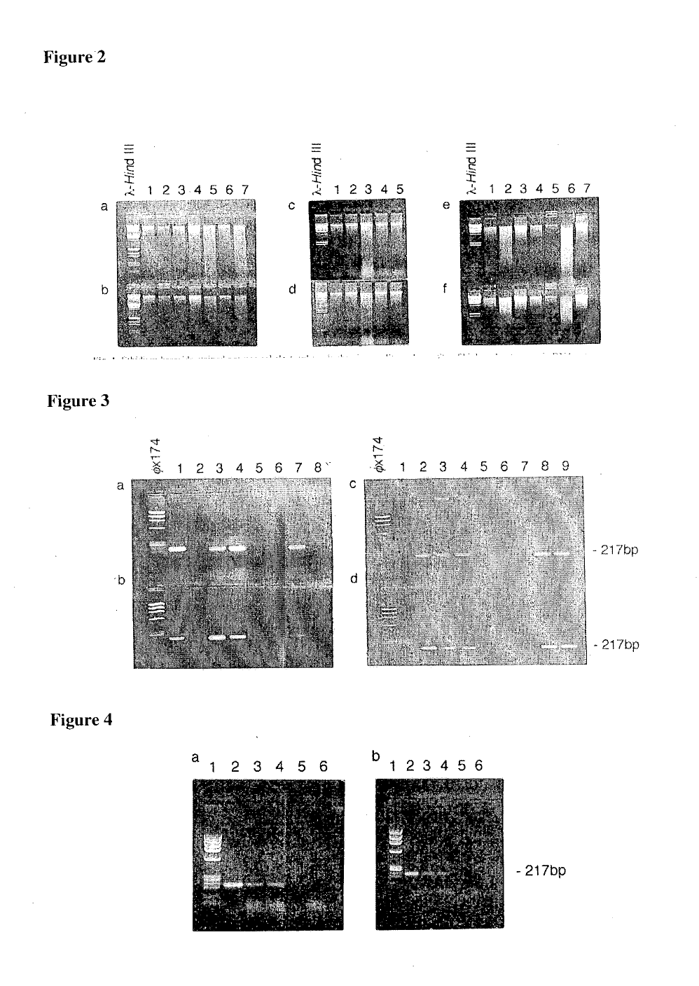

For HPV 16, 217-bp amplicons were detected with equal frequency in cervical scrapes and turnours whether collected by paper smear or by standard procedures (FIG. 2, Table 2). Similarly, the BRCAI and p53 gene amplifications in FNACs or in blood samples were equally successful with paper smear-derived and conventionally prepared DNA (Table 2 as shown below). Paper smears (>200) obtained by ordinary post, even during summer months when the temperature rose to 40-46° C. in India, generated the same results as standard procedures.

To evaluate the stability of the paper smear specimens for DNA elution and PCR amplification, some smears were stored for 1, 6 or 12 months. There was no discernible difference between DNA eluted or PCR amplifications obtained from smears stored for up to a year, whether at room temperature or at 4° C. (FIG. 3).

For additional molecular biology experiments, it was possible to extract a larger amount of DNA from the paper smears by using multiple pieces of the paper slides dropped into a micro centrifuge tube containing 100 μl of distilled water and processed for DNA extraction by a simple non-organic method. It was found that 5-7 μg of DNA could be extracted (>90% recovery) easily from a single paper smear and the DNA was suitable for PCR amplification, restriction endonuclease digestion and cloning or sequencing.

The similar work is been done in other systems also to establish the workability of the method of the instant Applications. The results are extremely encouraging. The details are as shown below in Table no. 2.

TABLE 2
Additional Genomes of Pathogenes Detected

| | | | | PCR + ve by | |
|---|---|---|---|---|---|
| | | Type of | No. of cases | standard | PCR + ve by |
| S.No. | Genomes/Genes | specimens | studied | PCR | boiling |
| 1. | C. trachomatis | Cervical swab | 50 | 15 (30.0%) | 15 (30.0%) |
| 2. | M. tuberculosis | Blood/ FNAC/ | 75 | 14 (18.6%) | 13 (17.3%) |
| | | Sputum | | | |
| 3. | M. pneumoniae | Nasal aspirates of | 75 | 22 (29.3%) | 22 (29.3%) |
| | | infants | | | |

Discussion

This study analysed >300 paper smears from various clinical specimens and obtained results that were identical to those obtained by conventional methods. Collection of cytological samples on small filter-papers and their dry shipment and storage at room temperature, coupled with a simple boiling or a non-organic method of DNA extraction, solves several disadvantage and reduces biohazards associated with the

TABLE 3
Quality of DNA and PCR positivity for HPV and other genes is different clinical
cytological specimens collected as paper smears or by standard procedures.

| | | | | Quality * of DNA and number of PCR- | | | |
|---|---|---|---|---|---|---|---|
| | Number | Genomes/ | | positive samples | | | |
| Type of | of | gene | Primer | Standard | PCR | Extraction | PCR |
| specimen | specimens | detected | location | extraction | positivity | by boiling | positivity |
| Cervical | 50 | HPV 16 | URR | +++ | +++ | +++ | +++ |
| scrape | | | | 50 | 14 | 50 | 14 |
| Cervical | 50 | HPV 16 | URR | +++ | +++ | +++ | +++ |
| tumour | | | | 50 | 39 | 50 | 39 |
| FNAC | 30 | BRCA 1 | Exon 2 | +++ | +++ | +++ | +++ |
| breast | | | | 30 | 30 | 30 | 30 |
| carcinoma | | | | | | | |
| Blood | 20 | P53 | Exon 5 | +++ | +++ | +++ | +++ |
| | | | | 20 | 20 | 20 | 20 |

* +++ indicates good quality DNA and PCR signal.

conventional handling of biological specimens and DNA extraction for large-scale population or epidemiological studies. The dried paper smear method is simple, rapid, safe and most convenient for collection, storage and transport of cervical scrapes/smears and biopsies and allows detection of HPV DNA or other gene sequences by PCR. The infectious hazard will also be reduced if pathogens present in clinical material do not survive drying and long-term storage. A larger amount of genomic DNA can be isolated from such dried paper smears if necessary, by using an extraction method with higher amounts of proteinase K at higher temperature (60-65° C.), after which the enzyme is auto-inactivated and autolysed [23,26]. This method is cost-effective compared with commercial extraction kits and is also rapid, as the entire procedure takes <3 h. It is performed in a single microcentrifuge tube, thus reducing mishandling or mislabelling and cross- contamination problems. A single paper smear is sufficient for as many as 10-12 PCR assays. This method offers a unique opportunity that a fraction of a cervical specimen can be employed as a paper smear and the rest could be processed for cytological Pap test or histopathology and the results can be compared with molecular diagnosis.

Dry storage of biological specimens on filter-paper at room temperature permits protection of DNA from degradation for a long period. In this study, dry smears were stored at room temperature for up to 1 year, with no alteration in the quality of DNA or subsequent PCR amplification (FIG. 3 and FIG. 4). The paper slides may be pre- treated with antibiotics and fungicides or denaturants to prevent growth of bacteria and other micro-organisms. This method can be universally employed for almost all types of biological specimens, such as cultured cells, cervical and buccal smears, blood, urine, amniotic/ascitic fluid, semen and bone marrow or needle aspirates, including solid tissue biopsy imprints.

Cervical scrapes and biopsies are often contaminated with blood and mucus, which might pose a potential problem for direct PCR. Erythrocyte contamination in cervical scrapes, particularly the porphyrin moiety of haemoglobin, acts as a strong inhibitor of PCR amplification [27]. Microwave treatment is capable of denaturing such inhibitory factors [28]; therefore, microwave treatment was used as an essential step before PCR amplification. Microwave treatment has also been shown to be efficient for elution of DNA from filter-paper blood spots [16] and to increase the sensitivity of PCR detection by about 2-3-fold [28]. Thus this method, which is a simple alternative to complicated conventional methods of collection, transport and storage of biological specimens, may be applied to population screening for a host of pathogens and genes, including for forensic purposes.

The method is being used routinely in this laboratory for diagnostic and screening purposes.

Although only dried blood spots on filter paper have so far been used to detect viral DNA sequences, no other cytological/clinical specimens such as cervical, oral/laryngeal scrapes, FNAC, urine, sputum semen and other bloody fluids, microbial cultures including solid tissue-biopsy imprints have been collected and stored in dried form in filter paper as “paper smear” in room-temperature (4 to 50° C.) for several years (15-20 years) to detect DNA sequences of pathogens or any other genes of interest by using a small piece (1 to 2 mn) of this sample-loaded filter paper directly into a single tube reaction for PCR amplification. This is for the first time that such a simple and inexpensive method has been developed for all variety of biological specimens. This will have tremendous implications in population screening of genes and pathogens as it overcomes all the problems of collection, transport and storage of biological specimens in ultra low temperature and biohazard and the cost associated with the standard DNA extraction procedure. Soon this method would replace the conventional method of population screening.

The invention of the instant Application is totally non-obvious from the prior art in several ways. The invention might appear to be simple. However, there are several aspects of the invention that contribute to the patentability of the instant invention. The dry form of the collection, storage, and transportation is never been tried previously, which would work on all kinds of specimens. Further, no technique is been found to have the workability using PCR. Further, there was no clue from any of the prior arts to help applicants to develop such a simple, rapid, and cost effective filter-paper method. It was only after several years to hard work that the applicants have developed an ideal process, which has such a wide application and can bring about paradigm shift in the way the screening is done in the world. This invention would have tremendous implications especially in underdeveloped and developing countries, where the infrastructure is a major limitation. Also, the efficacy of the methodology is exceptional. The results have been extremely encouraging with about 100% efficacy. This is further commendable because the workability is been established at temperature as high as about 50° C. This property would have extraordinary utility in tropical countries where temperature shoots up to very high levels. In addition, the samples can be stored for such long time duration as about 15 years. This is a reasonably high time duration. Now, if one looks into all these properties put together in one invention, then all the criteria of patentability like novelty, non-obviousness, and utility are established.

References

1. Luthra U K, Prabhakar A K, Seth P et al. Natural history of precancerous and early cancerous lesions of the uterine cervix. Acta Cytol 1987; 31: 226-234.

2. Das B C, Gopalkrishna V; Hedau S, Katiyar S. Cancer of the uterine cervix and human papillomavirus infection. Current Science 2000; 78: 52-63.

3. World Health Organization. Control of cancer of cervix uteri. A WHO Meeting. Bull World Health Organ 1986; 64: 607-618.

4. Howley P M. Role of the human papillomaviruses in human cancer. Cancer Res 1991; 51: 5019s-5022s.

5. zur Hausen H. Viruses in human cancers. Science 1991; 254: 1167-1173.

6. zur Hausen H. Papillomavirus infections—a main cause of human cancers. Biochem Biophys Acta 1996; 1288: F55-F78.

7. de Villiers E M. Heterogenicity of the human papillomavirus group. J Virol 1989; 63: 4898-4903.

8. zur Hausen H. Papillomavirus as carcinoma viruses. Adv Viral Gncol 1989; 8: 1-26.

9. Das B C, Sharma J K, Gopalkrishna Vet al. A high frequency of human papillomavirus DNA sequences in cervical carcino- mas of Indian women as revealed by southern blot hybridization and polymerase chain reaction. J Med Virol 1992; 36: 239-245.

10. Das B C, Murthy N S, Sharma J K et al. Human papillomavirus and cervical cancer in Indian women. Lancet 1989; 2: 1271.

11. Cuzick J, Terry G, Ho L, Hollingworth T, Anderson M. Human papillomavirus type 16 DNA in cervical smears as predictor of high-grade cervical cancer. Lancet 1992; 339: 959-960.

12. Das B C, Sharma J K, Gopalkrishna V; Luthra U K. Analysis by polymerase chain reaction of the physical state of human papillomavirus type 16 DNA in cervical preneoplastic and neoplastic lesions. J Gen Virol 1992; 73: 2327-2336.

13. Lie A K, Isaksen C V; Skarsvag S, Haugen G A. Human papillomavirus (HPV) in high-grade cervical intraepithelial neoplasia (CIN) detected by morphology and polymerase chain reaction (PCR): a cytologic correlation of 277 cases treated with laser conization. Cytopathology 1998; 10: 112-121.

14. Paavonen J, Koutsky L A, Kaviat N. Cervical neoplasia and other STD-related genital and anal neoplasia. In: Hohns K K, Mardh P A, Sperling P F, Weisner P J (eds) New York, McGraw Hill. Sexually transmitted diseases, 2nd edn. 1990: 561-592.

15. Panteleeff D D, John G, Nduati R et al. Rapid method for screening dried blood samples on filter paper for human immunodeficiency virus type I DNA. J Clin Microbial 1999; 37: 350-353.

16. Gupta B P, Jayasuryan N, Jameel S. Direct detection of hepatitis B virus from dried blood spots by polymerase chain reaction amplification. J Clin Microbiol 1992; 30: 1913-1916.

17. Kain K C, Lanar D E. Determination of genetic variation within Plasmodium falciparum by using enzymatically amplified DNA from filter paper disks impregnated with whole blood. J Clin Microbial 1991; 29: 1171-1174.

18. Cassol S, Salas T, Arella M, Neumann P, Schechter M T, O'Shaughnessy M. Use of dried blood spot specimens in the detection of human immunodeficiency virus type 1 by the polymerase chain reaction. J Clin Microbiol 1991; 29: 667-671.

19. Beebe J L, Briggs L C. Evaluation of enzyme-linked immuno-assay systems for detection of human immunodeficiency virus type I antibody from filter paper disks impregnated with whole blood. J Clin Microbiol 1990; 28: 808-810.

20. Garrick M D, Dembure B S, Guthrie R. Sickle-cell anemia and other hemoglobinopathies: procedures and strategy from screening employing spots of blood on filter paper as specimens. N Engl J Med 1973; 288: 1265-1268.

21. Bickel H C, Bachman C, Beacker R. Neonatal mass screening for metabolic disorders. Eur J Pediatr 1981; 137: 133-139.

22. Maeda M, Ito K, Arakawa H, Tsuija A. An enzyme-linked immunosorbent assay for thyroxine in dried blood spotted on filter paper. J Immunol Methods 1985; 82: 83-89.

23. Gopalkrishna V; Francis A, Sharma J K, Das B C. A simple and rapid method of high quality DNA isolation from cervical scrapes for detection of human papillomavirus infection. J Virol Methods 1992; 36: 63-72.

24. Sambrook J, Fritsch E F, Maniatis T. Molecular cloning. A laboratory manual, 2nd edn. Cold Spring Harbor, N.Y., Cold Spring Harbor Laboratory Press. 1989.

25. Kwok S, Higuchi R. Avoiding false positives with PCR. Nature 1989; 339: 237-238.

26. Jeanpierre M. A rapid method for the purification of DNA from blood. Nucleic Acids Res 1987; 15: 9611.

27. Higuchi R. Rapid, efficient DNA extraction for PCR from cells or blood. Amplifications 1989; 2: 1-3.

28. Cheryrou A, Guymarch'h C, Jasserand P, Blouin P. Improved detection of HBV DNA by PCR after microwave treatment of serum. Nucleic Acids Res 1991; 19: 4006.

1. A cytological specimen loaded filter paper useful for dry collection, transportation, and storage of cytological specimens at temperature ranging between 4° C. to 50° C. to screen for identifying gene sequences of pathogens using PCR.

2. A paper as claimed in claim 1, wherein the cytological specimens are selected from a group comprising cultured cells, cervical and buccal smears, scrapes, blood, urine, amniotic/ascitic fluid, semen, and bone marrow or needle aspirates, including solid tissue biopsy imprints.

3. A paper as claimed in claim 1, wherein the loaded-paper is stored in a sterile airtight bag.

4. A paper as claimed in claim 1, wherein the loaded-paper is workable for about fifteen years.

5. A paper as claimed in claim 1, wherein said paper is used for large scale screening especially for population from distant places.

6. A simple, rapid, safe, and cost-effective filter-paper method of dry collection, transportation, storage, and screening of cytological specimens at temperature ranging between 4° C. to 50° C. to screen for detection of genes responsible for infection using PCR with 100% efficacy, said method comprising the steps of:
i. applying the cytological specimen on to a pre-treated filter paper,
ii. drying the paper comprising the specimen,
iii. storing the paper of step (b) in a sterile airtight bag,
iv. adding small piece of the dried paper of step(c) into distilled water to obtain a solution,
v. boiling the paper price in a solution for a time duration ranging between 4-8 minutes,
vi. concentrating the solution to a range of one-fourth to one-fifth of the original volume to obtain a concentrate comprising DNA,
vii. adding PCR-mix to the concentrate,
viii. conducting direct-PCR on the concentrate, and
ix. screening for the infection using PCR product on electrophoresis.

7. A method as claimed in claim 6, wherein the method helps early identification of a disease condition.

8. A method as claimed in claim 6, wherein the filter paper is 3MM filter paper.

9. A method as claimed in claim 6, wherein boiling the solution for about 5 minutes.

10. A method as claimed in claim 6, wherein boiling is in microwave at about 360 W.

11. A method as claimed in claim 6, wherein DNA is stable in the paper for about fifteen years.

12. A method as claimed in claim 6, wherein said method can be used for large scale screening especially for population from distant places.

13. A method as claimed in claim 6, wherein about 90% of the DNA content is retrieved from the paper smears.

14. A method as claimed in claim 6, wherein DNA maintains its native form.

15. A method as claimed in claim 6, wherein the paper is pretreated with antibiotics, fungicides, and denaturants.

16. A method as claimed in claim 6, wherein said method can be employed for all types of biological specimens comprising cultured cells, cervical and buccal smears, scrapes, blood, urine, amniotic/ascitic fluid, semen, and bone marrow or needle aspirates, including solid tissue biopsy imprints.

17. A method as claimed in claim 6, wherein the bag is preferably a sealed polythene bag.
